(19) United States
(12) Patent Application Publication
(10) Pub. No.: US 2013/0115588 A1
(43) Pub. Date: May 9, 2013

(54) INTEGRATED BIOREACTOR AND SEPARATION SYSTEM AND METHODS OF USE THEROF

(71) Applicants: Kim Davis, Apex, NC (US); Guenko Guenev, North Kingstown, RI (US); Todd Benson, Apex, NC (US); James Kacmar, Apex, NC (US); John Moll, Apex, NC (US); Derrick Marconi, Apex, NC (US)

(72) Inventors: Kim Davis, Apex, NC (US); Guenko Guenev, North Kingstown, RI (US); Todd Benson, Apex, NC (US); James Kacmar, Apex, NC (US); John Moll, Apex, NC (US); Derrick Marconi, Apex, NC (US)

(73) Assignees: G & G TECHNOLOGIES, INC., North Kingstown, RI (US); SMARTFLOW TECHNOLOGIES, INC., Apex, NC (US)

(21) Appl. No.: 13/641,398

(22) PCT Filed: Apr. 15, 2011
(86) PCT No.: PCT/US11/32669

§ 371 Date: Jan. 14, 2013

Related U.S. Application Data
(60) Provisional application No. 61/324,695, filed on Apr. 15, 2010.
(60) Provisional application No. 61/325,024, filed on Apr. 16, 2010.

(51) Int. Cl. C12M 1/36

(52) U.S. Cl. CPC C12M 41/48
USPC 435/3; 435/286.1

(57) ABSTRACT

The present invention relates to methods and systems for culturing and purifying target substance(s), such as selected proteins, viruses, pathogenic bacteria, antibodies, antigens, clotting factors, glycoproteins, and hormones, from source liquids wherein the culturing and purification is effected in an integrated system including a bioreactor and at least one separation unit that functions under the control of a single operating system and provides for disposable components.

### CROSS-REFERENCE TO RELATED APPLICATIONS

The present application claims priority to U.S. Provisional Patent Application Ser. Nos. 61/324,695 filed on Apr. 15, 2010 and 61/325,024 filed on Apr. 16, 2010, the contents of each is incorporated herein by reference in its entirety for all purposes.

### BACKGROUND OF THE INVENTION

1. Field of Invention

The present invention relates to methods and systems for culturing and purifying target substance(s), such as selected proteins, viruses, pathogenic bacteria, antibodies, antigens, clotting factors, glycoproteins, and hormones, from source liquids wherein the culturing and/or purification is effected in an integrated system that functions under the control of a single operating system and provides for disposable components.

2. Related Art in the Field

Culturing of cells and separation systems are typically used for the growth of cells for biologicals, pharmaceuticals, and other cell-derived products of commercial value. Conventionally, cells have been attached to and grown on the interior surface of glass or plastic roller bottles or tubes or on culture plates. Further, product recovery and purification are complex procedures and usually involve multiple and separate units.

The expense of producing biologicals in bioreactors is exacerbated by the required cleaning, sterilization and validation of the standard stainless steel or glass bioreactors by the customer. Attempts have been made to solve this problem with the development of pre-sterilized components that need not be cleaned, sterilized or validated by end users. However, in some situations, pre-sterilization does not address the issue of pathogenic cultures and subsequent requirements for cleaning of such components.

Pathogenic animal viruses, such as the human immunodeficiency virus (HIV), the rabies and herpes viruses, and pathogenic bacteria such as Neisseria meningiditis and Mycobacteria avium must be studied with extreme precaution to avoid spread of the virus and contamination of workers and research areas. The problem is that in order to study these viruses, large quantities of viruses and large volumes of virus extracts must be prepared and isolated from growth media and contaminating cells, microbes and debris. Although other organisms, such as bacteria or yeasts do not usually require such large volumes of cell growth as are required for viruses to obtain sufficient material for study, the cells must also be cultured in quantity and handled with great care to avoid worker exposure and accidental release of the organisms. Further, proper cleaning and sterilization must be addressed before the system is reused.

Product harvesting is another area that great care must be taken to avoid worker exposure and accidental release of the organisms. Typically, harvesting includes filtration to separate, clarify, modify and/or concentrate a fluid solution, mixture or suspension. In the biotechnology and pharmaceutical industries, filtration is vital for the successful production, processing, and testing of new drugs, diagnostics and other biological products.

Different components are required to complete a process for culturing of cells, production of a target substance and separating of such target substance from the culture medium and heretofore, compiling components and having communication between such components required multiple operating systems. Often control involved constant surveillance of the systems components and any modification to the processes.

Thus, it would be advantageous to provide systems for culturing and/or separation of desired products, wherein the components of the systems are single use and disposable, easy to use, and inexpensive thereby enabling the preparation of desired product by people not specifically trained in aseptic techniques. Moreover, it would be advantageous to provide a system that is controlled and monitored by a single computer interphase that allows for additional components to be added to the system while maintaining control over all components.

### SUMMARY OF THE INVENTION

The present invention relates to systems for culturing and separating of cells or microbial organisms that is disposable, easy to use, inexpensive and versatile. The invention enables the preparation of target substances generated by cells or microorganisms to be grown and harvested safely, for a variety of purposes, without the need for specialized facilities such as temperature controlled rooms, laminar flow cabinets and sterilization equipment. Further, a significant amount of time is saved and used more efficiently when using single use and disposable components because the components can be pre-sanitized, ready to use without spending time to prepare or clean thereby saving on water usage and utilities. Still further, the consolidation of producing and separating a desired target substance into a single processing system eliminates the chance of cross-contamination or error. Notably, the possibility of contamination or spoilage is greatly reduced by avoiding movement of the product from machine to machine all of which may include different setup times, multiple delays and additional handling.

In one aspect, the system provides for an integrated system comprising a bioreactor vessel in fluid communication with at least one separation unit and under the control of a Human-Machine Interphase (HMI) for controlling processes during the culturing of cells or microorganisms in a culture medium and separating any target substance from such culture medium. The separation unit may include a filtration system, chromatography unit or a combination thereof.

The HMI includes integrated software to control the bioreactor and filtration and/or chromatography units to permit the user to observe and control all components from a single source spot. Further the software provides for the adding additional components with automatic recognition allowing for expansion of the system under a single controller.

In another aspect, the present invention provides for a bioreactor comprising a frame structure holding a disposable container for culturing microorganisms or cells comprising flexible or semi-flexible waterproof sheets fabricated to form a container. The disposable container can be fabricated in a multiplicity of different shapes including spherical, triangular, square, oblong, tubular, rectangular, or multifaceted and sized to fit within a bioreactor holder.

The bioreactor or disposable bioreactor unit comprises at least one inlet port for introducing gases or liquids and at least one exit port exhausting gases or for removing liquids. The at least one gas inlet port provides for input of gases including air, oxygen, carbon dioxide and/or nitrogen. Such ports may be adaptable or in fluid communication with control valves to monitor such input. The bioreactor or disposable bioreactor unit of the invention also may contain an inoculation port for introducing inoculants into the container. Either embodiment may further comprise a separate external chamber connected by tubing to the bioreactor or bioreactor disposable unit, for delivery of concentrated or dried growth medium, inoculum, or other substances.

In one aspect the present invention provides a culturing and separating system comprising:
a disposable culturing container for housing biomaterials for processing, wherein the disposable culturing container is positioned in a structure for supporting the disposable container;
a separating unit in fluid communication with the disposable culturing container, wherein the separating unit comprises at least one disposable filtration unit.

In yet another aspect, the present invention provides for a culturing and separating system comprising:
a disposable culturing container for housing biomaterials for processing, wherein the disposable culturing container comprises at least one input port, at least one exhaust port, at least one harvest port, a structure for supporting the disposable container, one or more sensors for sensing one or more parameters of the biomaterials in the container;
a separating unit in fluid communication with the disposable culturing container, wherein the separating unit comprises at least one disposable filtration unit; and
a monitoring means for operating and controlling conditions in the disposable culturing container and separation unit.

The system can further comprise sensors for monitor conditions within the system, wherein the sensors are connected to the bioreactor and/or the separating device and send signals to the HMI and can include, but not limited to, temperature, dissolved oxygen, pH, tank level, agitation speed, need for addition of substrate or nutrients, flow rate of gas or fluids including recirculation flow rate, inlet and outlet pressure, conductivity, turbidity, UV radiation, etc.

A still further aspect of the present invention provides for a method of producing and separating a target substance comprising the steps of:
introducing into a bioreactor a cell or micro-organism culture and culture medium fluid;
maintaining the bioreactor and cells or microorganism under conditions to assure the expression of the target substance;
moving a portion of the fluid, including cells or microorganisms and the target substance, through a separation device positioned in fluid communication with the bioreactor and separating therein at least some of the target substance from the fluid while allowing the remaining moving fluid and cells to pass through the separation device for return to the bioreactor; wherein the target substance separated from the fluid is removed to a collection vessel; and
operating and controlling the system and processes therein with a single computer having the ability to control and adjust parameters within all the components of the system.

Preferably, the bioreactor comprises a structural frame for holding a disposable container and the separation device is a disposable cross-flow filtration filter. In such a disposable system, other disposable units can include the following but not limited to pump head, flow meter, pressure transducer, process lines and connectors between the bioreactor and separation unit.

Other features and advantages of the present invention will be better understood by reference to the drawings and detailed description that follows.

### BRIEF DESCRIPTION OF THE FIGURES

FIG. 1 is a schematic diagram showing components of the present invention including a bioreactor unit, filtration unit in communication with computer module.

FIG. 2 is a schematic diagram showing components of the present invention including a bioreactor unit, chromatography unit in communication with computer module.

FIG. 3 is a schematic diagram showing components of the present invention wherein the bioreactor unit includes a disposable container and the separation device includes a disposable filtration unit.

FIG. 4 is a schematic diagram showing components of the present invention wherein the bioreactor unit includes a disposable container and the separation device includes a disposable chromatography unit.

FIG. 5 is a photograph of one embodiment of the system of the present invention

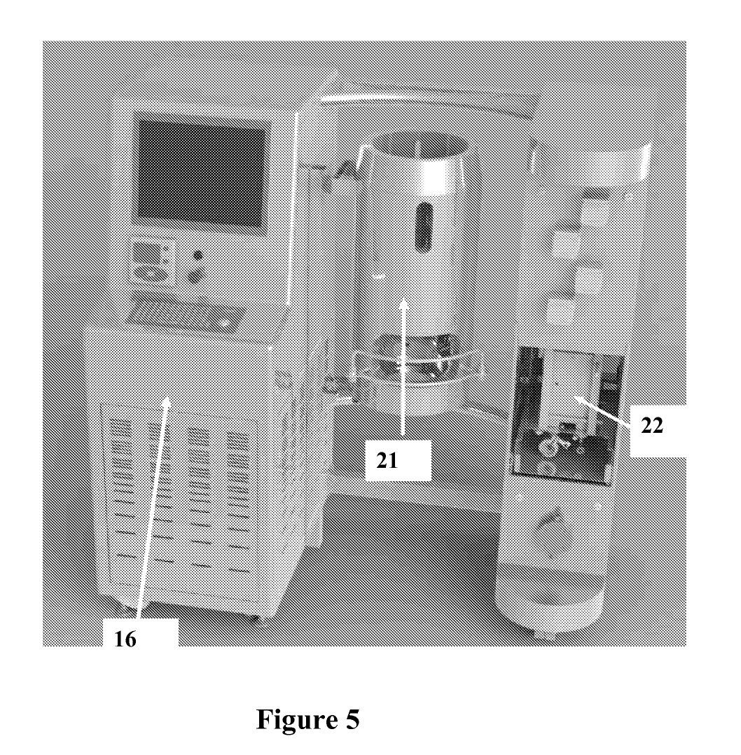

FIG. 6 is a photograph of additional units that can be added to the basics of the system for combining a microfiltration, ultrafiltration or nanofiltration unit or in the alternative an additional chromatography unit, all of which can be controlled by a single computer.

FIG. 7 shows examples of applicable permeate and retentate sheets used in the stack filter cassette of the present invention.

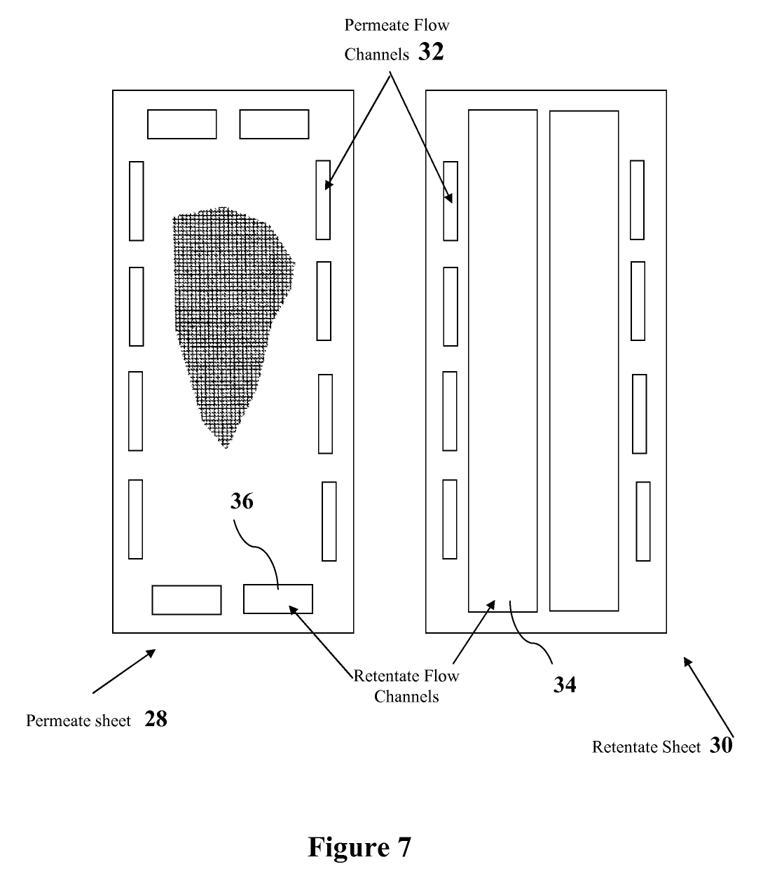

FIG. 8 shows an example of an applicable separation retentate sheet used for a series-flow configuration as shown in FIG. 12.

FIG. 9 shows the setup of filter end plates to provide for a series-flow configuration and further shown in FIG. 11.

FIG. 10 shows a screen shot of the HMI system that provide control over operation and modification of the different components included in the functioning of the bioreactor/separation system, whether non-disposable or single use-disposable, of the present invention.

FIG. 11 shows a series-flow configuration including the filter end plates of FIG. 9 in combination with at least the retentate separation sheet of FIG. 8.

FIG. 12 shows a series of possible units that may be added on to the Bioreactor vessel for further clarification and separation of product, all of which are recognized and immediately controlled by the HMI system.

FIG. 13 shows the components of a preferred cross-flow filtration system of the present invention.

### DETAILED DESCRIPTION OF THE INVENTION

### Definitions

In the description of the present invention, certain terms are used as defined below.

“Source liquid” as used herein refers to a liquid containing at least one and possibly two or more target substances or products of value which are sought to be purified from other substances also present. In the practice of the invention, source liquids may for example be aqueous solutions, organic solvent systems, or aqueous/organic solvent mixtures or solutions. The source liquids are often complex mixtures or solutions containing many biological molecules such as proteins, antibodies, hormones, and viruses as well as small molecules such as salts, sugars, lipids, etc. Examples of source liquids that may contain valuable biological substances amenable to the purification method of the invention include, but are not limited to, a culture supernatant from a bioreactor, a homogenized cell suspension, plasma, plasma fractions, milk, colostrum and cheese whey.

“Target substance” as used herein refers to the one or more desired product or products to be purified from the source liquid. Target substances are typically biological products of value, for example, immunoglobulins, clotting factors, vaccines, antigens, antibodies, selected proteins or glycoproteins, peptides, enzymes, etc. The target substance may be present in the source liquid as a suspension or in solution. For convenience, the term “target substance” is used herein in the singular, but it should be understood that it may refer to more than one substance that is to be purified, either together as co-products or separately (e.g., sequentially) as discrete recovered components.

“Cross-flow filter” as used herein refers to a type of filter module or filter cassette that comprises a porous filter element across a surface of which the liquid medium to be filtered is flowed in a tangential flow fashion, for permeation through the filter element of selected component(s) of the liquid medium. In a cross-flow filter, the shear force exerted on the filter element (separation membrane surface) by the flow of the liquid medium serves to oppose accumulation of solids on the surface of the filter element. Cross-flow filters include microfiltration, ultrafiltration, nanofiltration and reverse osmosis filter systems. The cross-flow filter may comprise a multiplicity of filter sheets (filtration membranes) in an operative stacked arrangement, e.g., wherein filter sheets alternate with permeate and retentate sheets, and as a liquid to be filtered flows across the filter sheets, impermeate species, e.g. solids or high-molecular-weight species of diameter larger than the filter sheet's pore size, are retained and enter the retentate flow, and the liquid along with any permeate species diffuse through the filter sheet and enter the permeate flow. In the practice of the present invention, cross-flow filtration is a preferred separation method. Cross-flow filter modules and cross-flow filter cassettes useful for such filtration are commercially available from Smartflow Technologies, Inc. (Apex, N.C.). Suitable cross-flow filter modules and cassettes of such types are variously described in the following United States patents: U.S. Pat. No. 4,867,876; U.S. Pat. No. 4,882,050; U.S. Pat. No. 5,034,124; U.S. Pat. No. 5,034,124; U.S. Pat. No. 5,049,268; U.S. Pat. No. 5,232,589; U.S. Pat. No. 5,342,517; U.S. Pat. No. 5,593,580; and U.S. Pat. No. 5,868,930; the disclosures of all of which are hereby incorporated herein by reference in their respective entireties.

“Chromatography resin” as used herein refers to a solid phase that selectively or preferentially binds one or more components of the source liquid. In the practice of the invention, such “chromatography resins” can be selected from any of the groups of resins commonly described as affinity, ion exchange and ion capture resins. The resins need only possess an associated ligand that will selectively or preferentially capture a substance of interest from the source liquid. Useful chromatography resins typically comprise a support and one or more ligand(s) bound thereto that provide(s) the selective or preferential binding capability for the target substance(s) of interest. Useful supports include, by way of illustrative example, polysaccharides such as agarose and cellulose, organic polymers such as polyacrylamide, methylmethacrylate, and polystyrene-divinylbenzene copolymers such as for example Amberlite® resin, commercially available from Rohm & Haas Chemical Co., Philadelphia, Pa. It should be recognized that although the term “resin” is commonly used in the art of chromatography, it is not intended herein to imply that only organic substrates are suitable for resin substrate use, since inorganic support materials such as silica and glasses have utility as well.

In the practice of the present invention, the resin may be in the form of beads which are generally spherical, or alternatively the resin may be usefully provide in particulate or divided forms having other regular shapes or irregular shapes. The resin may be of porous or nonporous character, and the resin may be compressible or incompressible. Preferred resins will be physically and chemically resilient to the conditions employed in the purification process including pumping and cross-flow filtration, and temperatures, pH, and other aspects of the liquids employed. The resin as employed in the practice of the present invention is preferably of regular generally spherical shape, nonporous and incompressible.

“Affinity ligand” as used herein refers to a moiety that binds selectively or preferentially to a component of the source liquid through a specific interaction with a binding site of the component. In the practice of the invention, the affinity ligand is typically immobilized to a solid phase such as a resin. Examples of affinity ligands that can be bound to the resin support to provide chromatography resins useful in the process of the present invention include: protein A and protein A analogs, which selectively bind to immunoglobulins; dyes; antigens, useful for purification of associated antibodies; antibodies, for purification of antigens; substrates or substrate analogs, for purification of enzymes; and the like. Affinity ligands and methods of binding them to solid support materials are well known in the purification art. See, e.g., the reference texts Affinity Separations: A Practical Approach (Practical Approach Series), Paul Matejtschuk (Editor), Irl Pr: 1997; and Affinity Chromatography, Herbert Schott, Marcel Dekker, New York: 1997.

“Affinity chromatography resin” or “affinity resin” as used herein refers to a chromatography resin that comprises a solid support or substrate with affinity ligands bound to its surfaces. Illustrative, non-limiting examples of suitable affinity chromatography resins include spherical beads with affinity ligands bound to the bead surfaces, wherein the beads are formed of cellulose, polystyrene-divinylbenzene copolymer, polymethylmethacrylate, or other suitable material. Preferred are rigid, non-porous cellulose beads with bound affinity ligands. An illustrative particularly preferred embodiment employs “Orbicell®” beads (commercially available from Accurate Polymers, Inc., Highland Park, Ill.) that can be covalently coupled, e.g., by well-known methods within the skill of the art, to suitable affinity ligands, e.g. Protein A.

“Ion exchange chromatography resin” or “ion exchange resin” as used herein refers to a solid support to which are covalently bound ligands that bear a positive or negative charge, and which thus has free counterions available for exchange with ions in a solution with which the ion exchange resin is contacted.

“Cation exchange resins” as used herein refers to an ion exchange resin with covalently bound negatively charged ligands, and which thus has free cations for exchange with cations in a solution with which the resin is contacted. A wide variety of cation exchange resins, for example, those wherein the covalently bound groups are carboxylate or sulfonate, are known in the art. Commercially available cation exchange resins include CMC-cellulose, SP-Sephadex®, and Fast S-Sepharose® (the latter two being commercially available from Pharmacia).

“Anion exchange resins” as used herein refers to an ion exchange resin with covalently bound positively charged groups, such as quaternary amino groups. Commercially available anion exchange resins include DEAE cellulose, QAE Sephadex®, and Fast Q Sepharose® (the latter two being commercially available from Pharmacia).

“Cell-culturing,” as used herein refers to culturing cells by a method which includes controlling the cell density of a cell culture, controlling the cell activity of a cell culture, or controlling both the cell density of a cell culture and the cell activity of the cell culture. “Cell activity,” as that term is used herein, means production rate by cells of cell products such as, for example, viruses, proteins expressed by recombinant DNA molecules within the cells, natural proteins, nucleic acids, etc.

The present invention comprises a culturing system in which a desired product may be grown to high concentrations in an open or closed system using a bioreactor and separation module.

After the appropriate number of target substance has been produced, the target substance is separated from the host cells, growth medium constituents and unwanted growth products for subsequent concentration and/or removal from the system.

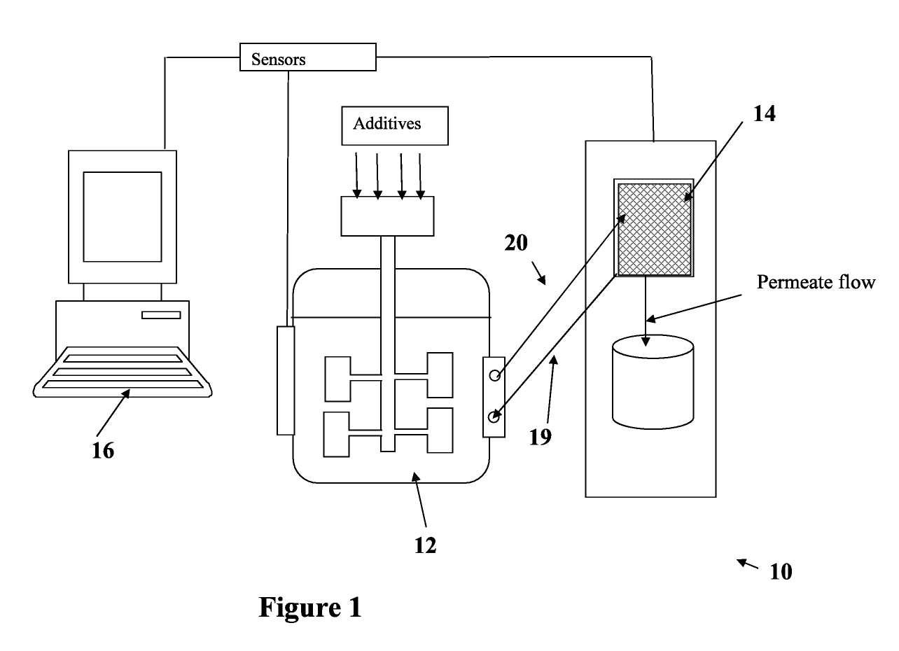

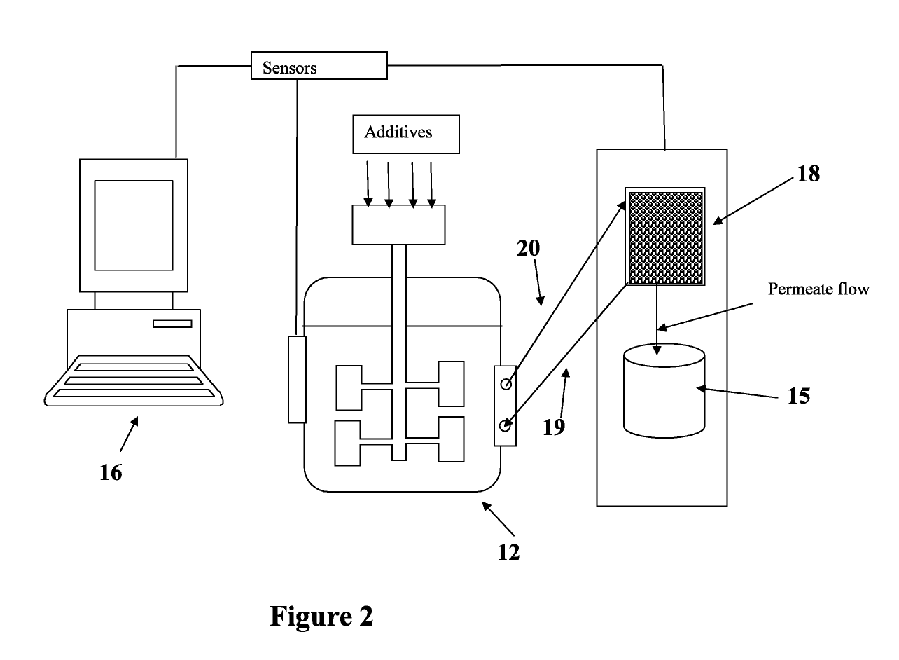

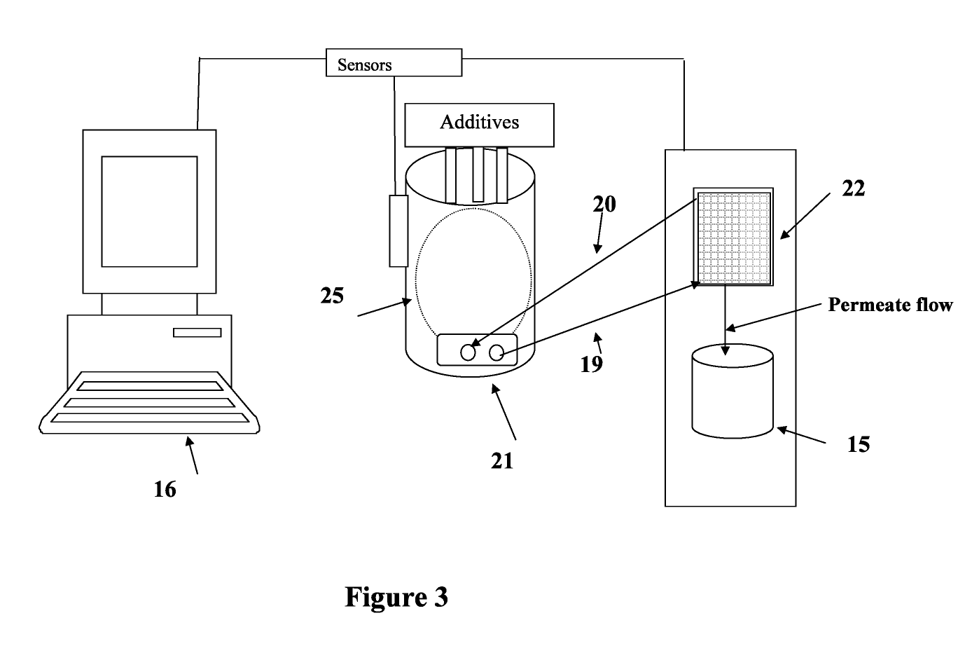

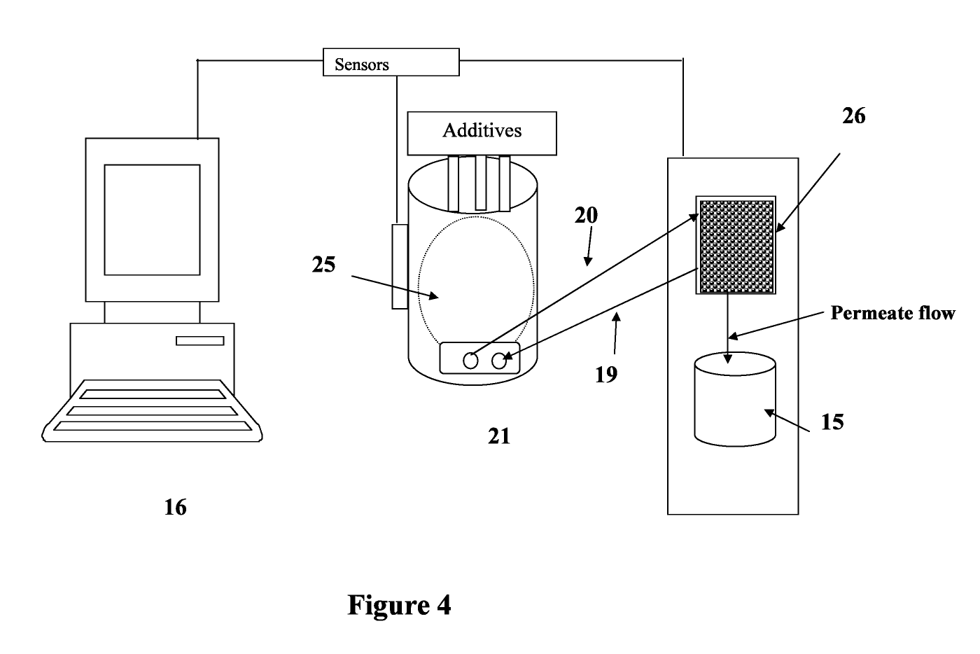

In embodiments as shown in FIGS. 1 and 2, the bioreactor 12 is vessel of stainless steel, glass or other durable easily sterilizable material capable of holding a sufficient quantity of materials to meet commercial needs including about 5 to 5000 liters of medium. Such reservoir may be of a type commercially available from the G&G Technology, North Kingston, R.I. Other cell-culture reactor types include airlift reactors, Carberry-type reactors, loop reactors, etc. In FIGS. 3 and 4 the bioreactor 21 includes a frame structure for holding a single use disposable liner or bag, and such a disposable bioreactor unit is commercially including the G & G Omni Bioreactor System from G&G Technology, North Kingstown, R.I.

At least one variable speed pump is connected to the bioreactor or disposable bioreactor unit for moving fluids into and out of the vessel and for moving fluid to and from the separation unit. The pump may comprise a peristaltic pump, a variable speed gear pump, or a positive displacement rotary lobe pump. The control of the pumps is monitored by controlling systems within the computer module 16, as shown in FIGS. 1, 2, 3 and 4. Movement of fluids may also be accomplished by such transfer methods as pressure, squeeze-transfer, gravity, etc.

The bioreactor vessel 12 or disposable bioreactor unit 21 may be equipped with stirring or agitation means to promote uniform distribution of medium components. The stirring or agitation means may include impellers, plungers, magnetic and non-magnetic devices, paddles, bladders, and pumping devices that can be located at any location of the bioreactor vessel or disposable container and drive by means including mechanical, pneumatically and/or hydraulically. For example, a magnetic stirrer, an internal agitator, or a bottom mounted, magnetically coupled agitator made be used and commercially available from APCO Technologies (Vancouver, Wash.).

In the alternative, the bioreactor vessel can be disposed on a magnetic stirrer device which provides agitation of the contents within the vessel. The magnetic stirrer may suitably be of a type having a variable speed, to provide a varying level of agitation in the vessel depending on the density and suspension characteristics of the nutrient medium contained therein.

The bioreactor or disposable bioreactor unit may also be provided with a medium pH monitoring and adjustment means or other means for adjustment of medium components or conditions of incubation according to means and devices known in the art.

The bioreactor or disposable bioreactor unit may be associated with a temperature control unit, may be placed in a controlled temperature environment or may be left at ambient temperature and varied depending on the desired cell growth conditions.

As shown in FIGS. 3 and 4, the disposable bioreactor unit 21 comprises a support housing wherein the interior chamber of the support housing is lined with a disposable container and sealed with a head plate to form a sealed chamber and may be used in a vertically oriented bioreactor. Although this system provides a disposable liner, the head plate and the interior chamber will still require cleaning and sterilization. As such, the disposable container can be a closable bag with inlet and outlet ports which allows for not only vertical but also horizontal placement of the disposable container. The disposable container is suitable for on-site use in small scale production of microorganisms, cells or expressed target substances. Preferably, the disposable container is inexpensive, easy to use, fabricated from a material that is sufficiently strong to allow the scale-up of culture size and allows for the culturing of a wide range of cells under both aerobic and anaerobic conditions and/or under dark or light conditions.

The disposable liner or bag can be fabricated from any material that does not interact with the components contacting such surfaces of the liner or bag. Preferably, the liner or bag is fabricated from a presterilized flexible, disposable, gas impermeable or gas permeable polymeric or cellulose containing material. Any commercially available disposable bag can be used in the present system, for example disposable bags available from Hyclone, Sartorius, Pall, Parker-Mitos, etc.

The disposable liner or bag 25 as shown in FIGS. 3 and 4 may be fabricated with a plurality of layers such as the multilayered fabric construction used in the synthesis of custom bags manufactured by Newport Biosystems, Inc. (Anderson, Calif.). A typical four-ply fabric construction would have individual layers, sequentially from the outer bag layer, of nylon, polyvinyldichloride (PVDC), a linear low density polyethylene (LLDPE), and a LLDPE inner layer for contacting the cells and the biological media. The plies of the bag have thicknesses with physical and molecular properties to provide the desired puncture strength, tensile strength, flexural strength, cell and gas and liquid permeabilities in appropriate ranges, and weldability and/or bondability or fusibility. The disposable liner or bag may be made to hold anywhere from milliliters of fluid to thousands of liters of fluid.

The shape of the liner or bag is determined by the size and shape of the bioreactor support structural, although a preferred embodiment has an approximately cylindrical shape with rounded corners to overcome any deadspace. However, any other shape is applicable that will closely fit the size and shape of the interior of the bioreactor support structure when the disposable liner or bag is filled.

To assist in monitoring the reaction activity and/or components within the bioreactor vessel, whether disposable or non-disposable, the vessel is communicatively connected to instrumentation that monitors temperature, oxygen contents, pH, tank level, any agitation speed, need for additional substrates or nutrients, gas flow rate, etc. Further flow meters, with control valves, may be in communication with the bioreactor vessel and computer module to monitor the input of gases, such as air, oxygen, carbon dioxide, nitrogen or any other gas necessary for culturing of cells or microorganisms. Feedback control of such components is linked to the computer module 16.

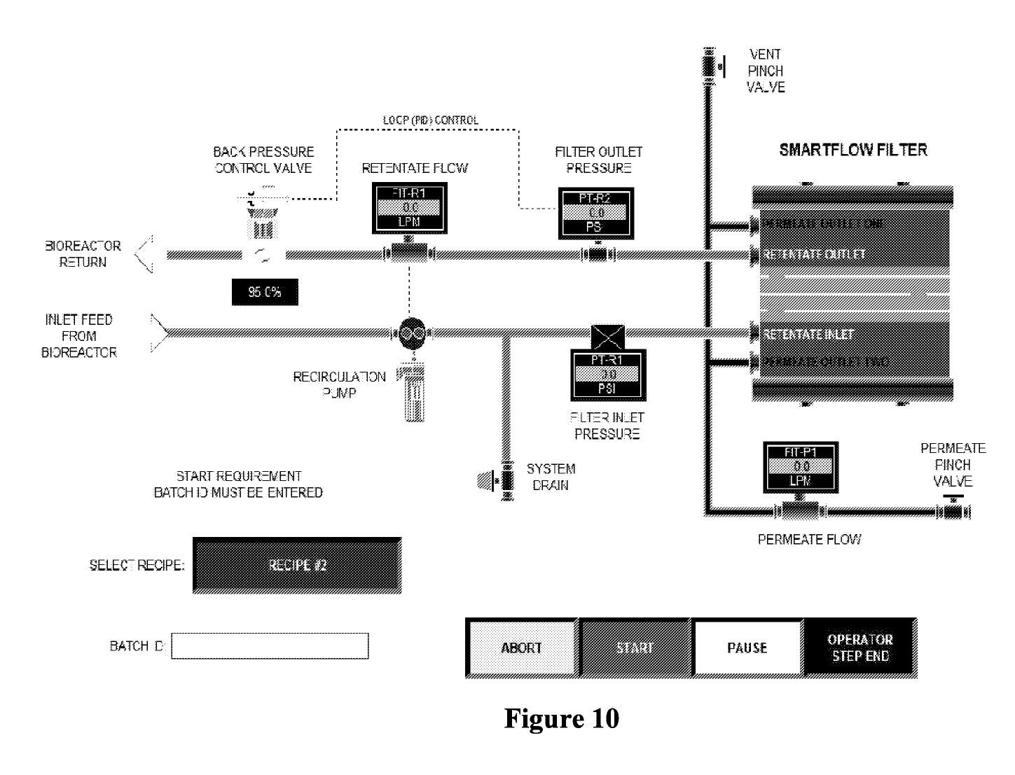

The instrumentation is monitored by the computer and adjustments can be made to the system when needed. FIG. 10 is a screen shot indicating the components that can be monitored during the culturing and separation process including filter inlet and outlet pressure, flow from the bioreactor to the filter system, circulation of the retentate to and from the filter system, speed of the circulation pump, activation of valves, among other parameters to ensure optimal separation of a target substance.

A proportional-integral-derivative controller (PID controller) may be used as part of the control system. The PID controller has the ability to control recirculation rate, pressure, tank level, temperature, oxygen content, agitation speed, pH, amount of additives, gas flow rates and other necessary parameters. The PID controller calculations use an algorithm performed by the computer system and involve three separate parameters, and is accordingly sometimes called three-term control: the proportional value determines the reaction to the current error, the integral value determines the reaction based on the sum of recent errors, and the derivative value determines the reaction based on the rate at which the error has been changing. The weighted sum of these three actions is used to adjust the process via a control element such as the position of a control valve or the power supply of a heating element. By tuning the three constants in the PID controller algorithm, the controller can provide control action designed for specific process requirements.

The bioreactor vessel, whether disposable or non-disposable, can include one or more gas removal means or fill means incorporated into the bioreactor vessel or disposable liner or bag assembly. For example, as medium is pumped into the bag, gas will be displaced and must have a release mechanism. There are a variety of means known in the art for releasing gas from media storage bags as the bags are filled with media. Similar gas releasing means will work in the present invention. In the alternative the media is conditioned before it is pumped into the bioreactor vessel or disposable bag. Conditioned media has been gassed to contain desired quantities of oxygen and/or other gases.

All ports, such as input and output ports, whether for gas, tissues, microorganisms, culture media or sensor instrumentation can be disposable, and thus, disposable along with a disposable culture bag or liner after use in the system.

Medium can be recirculated through the bioreactor vessel, whether disposable or non-disposable and fresh medium can be added to the system. The medium can be any fluid suitable for culturing cells. Many such media are known in the art. Examples of media suitable for culturing animal cells include: hormonally-defined media; serum-supplemented basal media, such as Dulbecco's Modified Eagle's Basal Medium; etc. Examples of culture media suitable for culturing microbes include well-defined media, undefined complex media, etc. Nutrients can be introduced to medium at any suitable point in the path of flow of medium between the bioreactor and separation unit. Suitable nutrients can be any nutrients suitable for culturing cells in the system and may include, for example, yeast extract, amino acids, sugar, salt, vitamins, etc.

The medium can be included in a separate vessel and introduced into the bioreactor vessel when needed through tubing conduits formed of suitable tubing, such as glass, ceramic, stainless steel or other metal, polymers such as Teflon polytetrafluoroethylene, rubber, etc.

In one embodiment, the bioreactor vessel, whether disposable or non-disposable, may include biocompatible macroporous ceraminc particles or plates having pores of sufficient size for positioning of any cells or microorganisms therewithin.

Cell waste and target substance can be moved from the bioreactor and transported to the separation unit as shown in FIGS. 1, 2, 3 and 4 for separation of the target substance produced by the cell or microorganism from the medium.

An example of a suitable separation unit is a chromatographic column (CC), as shown in FIGS. 2 and 4, for recovery of a desired target substance. The source liquid from the bioreactor vessel , whether disposable 21 or non-disposable 12, is transferred to a separation device, disposable 26 or non-disposable 18 wherein it is contacted with a chromatography resin, which selectively or preferentially binds the target substance. It is possible to add the chromatography resin to the CC already containing the (optionally clarified) source liquid, or alternatively the chromatography resin may be charged to the CC and the source liquid thereafter added. The transfer of the source liquid to the CC may be carried out in any other suitable manner, e.g., in a batch, semi-batch or continuous manner.

Suitable chromatography resins for use in this step may be in the form of beads or other particulate or finely divided forms capable of binding the target substance. The chromatography resin can be selected from any of the groups of resins commonly described as affinity, ion exchange and ion capture resins, and a wide variety of resins of such types is readily commercially available. The resins possess a chemistry or ligand chemistry that will capture the substance of interest and bind the target substance to the resin. A particularly useful chromatography resin is provided in the form of uniformly spherical, non-porous, rigid, non-agglomerating, particles that are in the range of about 0.1 to 1,000 microns in size and have a low affinity for nonspecific binding. In one particularly preferred embodiment of the invention, the chromatography resin comprises cellulose beads, 1 to 3 microns in diameter, with Protein A ligands covalently bound to its surface. Such beads are highly useful in the purification of monoclonal antibodies from tissue culture and mouse ascites fluid. Beads of such type are commercially available under the trademark “Orbicell®” from Accurate Polymers, Inc. (Highland Park, Ill.).

The source liquid is incubated with the chromatography resin for a sufficient contact time to lead to binding of a desirably high percentage of the target substance to the chromatography resin, and to form resulting resin-target complexes. A simple method of incubation may entail stirring or shaking the separation device containing the slurry. The preferred contact (incubation) time in the separation device depends on the particular chromatography resin employed and its concentration of binding sites for the target substance, as well as the relative concentration of beads and target substance. The reaction time of the chemistry will vary from ligand to ligand, but the higher the concentration of available binding sites compared to the target substance, the shorter the preferred incubation time. Temperature may be controlled during the incubation step by the thermal jacket (or other heat transfer means) to provide the liquid and resin mixture with a suitable temperature to preserve the target substance's activity. Suitable temperatures for such purpose may be readily determined within the skill of the art and without undue experimentation.

For separation of the target substance from the resin, the resin may be diafiltered against a diafiltrate liquid which is selected to the specific target-resin complexes and the diafiltrate containing the target substance is captured in a second reservoir 15 wherein the target substance may be concentrated to a useful concentration.

Clearly, there can be several clarification steps to remove from the source liquid particulate contaminants prior to sending the source liquid into the separation unit for concentration of the target substance to avoid contaminating the purified target substance. The clarification step can be accomplished by methods well-known in the purification art, for example, centrifugation, gravity separation, precipitation, flocculation-assisted sedimentation, decanting, normal filtration, sieving, absorption, adsorption and tangential flow filtration.

Alternatively, the source liquid may already be sufficiently clean to make this step unnecessary.

Additionally it should be recognized that retentate leaving the filtration system does not need to be reintroduced into the bioreactor vessel and accommodations may be made for an additional retentate vessel for storage of the retentate that can be recirculated into the filtration system providing for additional removal of any target substance.

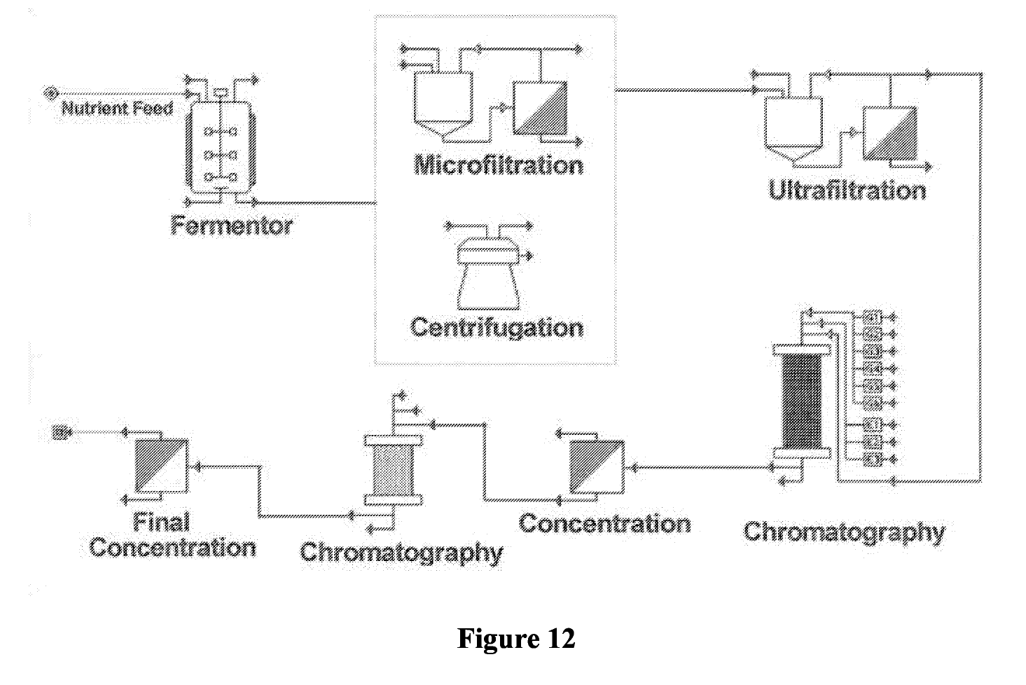

As shown in FIG. 12, it is evident that multiple add-on units can be included in the system. For example source fluid leaving the bioreactor vessel or fermentor can be initially purified through a clarification step and such clarified solution can be introduced into an ultrafiltration unit and multiple other units in a step-by-step fashion for production of the final product. Importantly as each additional unit is added to the overall system, the present integrated system can be customized for the specific process of interest and the HMI easily recognizes each new unit with immediate control of the movement of fluids from one unit to the next unit.

Optionally, the contaminants and excess liquid are separated and dialyzed away from the chromatography resin, now bound to the target substance, by means of a separating cross-flow filter module. The optimal separating cross-flow filter module preferably has a membrane pore size that is 1.5 to 10 times smaller than the mean diameter of the chromatography resin beads. The channel height of the separating cross-flow filter module is desirably 1.2 to 10 times larger than the mean diameter of the chromatography resin beads to provide satisfactory clearance and efficient hydrodynamic behavior of the filter module. A highly preferred design of the separating cross-flow filter module is an open channel module with even distribution of flow to the retentate channels. A cross-flow filter module suitable for this purpose is commercially available from Smartflow Technologies, Inc. (Apex, N.C.). The resin slurry can be recirculated across the cross-flow filter module for separation therein and retentate liquid is returned to either the bioreactor vessel or separation device.

It should be appreciated that a number of alternative apparatus arrangements may be constructed, arranged and operated, to carry out the separation method of the present invention in various embodiments thereof.

Another preferred separation device is a filtration unit, including either a disposable or non-disposable unit, and can be selected from a tangential flow filtration or direct flow/dead end filtration device. Tangential flow filtration (TTF) is different from dead end filtration in which the feed is passed through a membrane or bed, the solids being trapped in the filter and the filtrate being released at the other end. Tangential flow filtration gets its name because the majority of the feed flow travels tangentially across the surface of the filter, rather than into the filter. The principle advantage of this is that the filter cake (which can bind-up the filter) is substantially washed away during the filtration process, increasing the length of time that a filter unit can be operational. TTF can be a continuous process, unlike batch-wise dead-end filtration.

A tangential flow device may comprise a mass transfer culture system utilizing a hollow fiber device as marketed by Amicon Corporation (Danvers, Mass.) or Microgon Corp. (Laguna Hills, Calif.), hollow ceramic devices by Pall Corporation or plate and frame devices such as Minitan® or Pellicon Cassette®(Millipore Corp., Bedford, Mass.). Thus, a hollow fiber device such as the stainless steel Microgon® equipped with a 0.2 micron hydrophilic membrane may be used for small to medium volume (1000 ml) applications.

A preferred filter system component that may be used in the present invention is disclosed in the following United States patents: U.S. Pat. No. 4,867,876; U.S. Pat. No. 4,882,050; U.S. Pat. No. 5,034,124; U.S. Pat. No. 5,034,124; U.S. Pat. No. 5,049,268; U.S. Pat. No. 5,232,589; U.S. Pat. No. 5,342,517; U.S. Pat. No. 5,593,580; and U.S. Pat. No. 5,868,930, referred to above, and comprises stacked filter plates forming a cross-flow filter and is capable of substantially uniform transverse distribution of inflowing liquid from a feed port and highly uniform liquid cross-flow across the full transverse extent of the flow channel. Useful cross-flow filters include microfiltration, ultrafiltration, nanofiltration, supermicrofiltration and reverse osmosis filter systems.

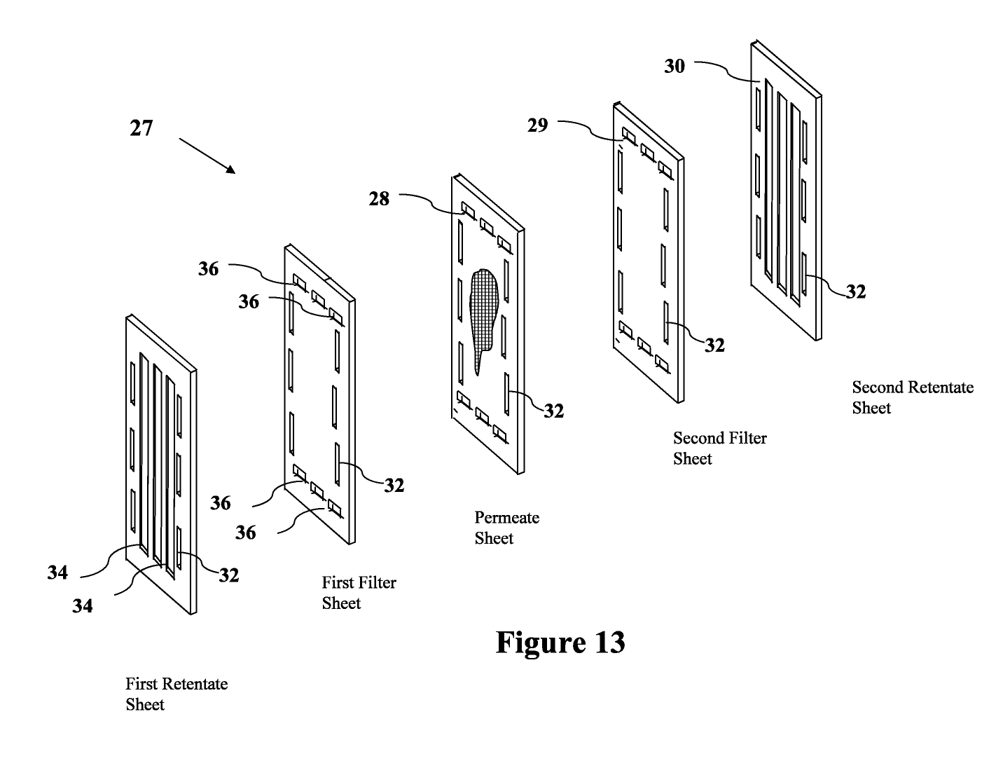

Thus, the cross-flow filtration unit comprises a multilaminate array 27, as shown in FIG. 13, of sheet members of generally rectangular and generally planar shape with main top and bottom surfaces, wherein the sheet members include in sequence in the array a first retentate sheet 30, a first filter sheet 29, a permeate sheet 28, and second filter sheet 29, and a second retentate sheet 30, wherein each of the filter and permeate sheet members in the array has at least one inlet basin opening 36 at one end thereof, and at least one outlet basin opening 36 at an opposite end thereof, with at least one permeate passage opening 32 at longitudinal side margin portions of the sheet members; each of the first and second retentate sheets having at least one channel opening 34 therein, wherein each channel opening extends longitudinally between the inlet and outlet basin openings of the sheets in the array and is open through the entire thickness of the retentate sheet, and with each of the first and second retentate sheets being bonded to an adjacent filter sheet about peripheral end and side portions thereof, with their basin openings and permeate passage openings 32 in register with one another, and arranged to permit flow of filtrate through the channel openings of the retentate sheet 34 between the inlet 36 and outlet basin 36 openings to permit flow through the filter sheet to the permeate sheet and then on to the permeate passage openings.

According to one embodiment of the present invention, a cross-flow filtration module with uniform geometry is utilized for conducting the membrane separation. The phrase “uniform geometry” is defined herein as the geometric structure of a cross-flow filtration module, characterized by at least one permeate flow passage, at least one inlet, at least one outlet, and multiple fluid-flow sub-channels that are of substantially equal length between the inlet and the outlet.

In a preferred embodiment of the present invention, cross-flow filtration modules with sub-channels that are equidistant to the inlet and outlet of said modules are employed for membrane separation. Moreover, such cross-flow filtration modules are characterized by optimal channel height, optimal transmembrane pressure, optimal membrane pore size and pore structure, optimal membrane chemistry, etc., which characteristics are selected in order to achieve the best combination of product quality and production yield.

For example, shear at the surface of the membrane is critical in minimizing gel layer formation, but excessive shear is deleterious in the following three key aspects: (1) excessive shear increases energy consumption, (2) excessive shear interferes with diffusion at the membrane surface, upon which the separation process directly depends, (3) excessive shear can deprive certain compounds of their bioactivities. It therefore is desirable to maintain shear within an optimal range.

Furthermore, it is possible to optimize the separate processes with cross-flow filtration modules of variable channel velocities but of uniform channel heights, given the fact that most commercial cross-flow modules are only available in a single channel height.

The transmembrane pressure (TMP) of the cross-flow filtration membrane can also be optimized after the appropriate tangential velocity has been determined. Transmembrane pressure is calculated as TMP=(inlet pressure+outlet pressure)/2−permeate pressure. The purpose of optimizing the transmembrane pressure is to achieve maximum permeate flow rate. The normal relationship between transmembrane pressure and permeate flow rate can be best represented by a bell curve. Increases in transmembrane pressure cause increases in the permeate rate, until a maximum is reached, and after which any further increases in transmembrane pressure result in decreases in the permeate rate. It is therefore important to optimize the transmembrane pressure so that the maximum permeate flow rate can be obtained.

The cross-flow filter may comprise a multiplicity of filter sheets (filtration membranes) in an operative stacked arrangement, e.g., wherein filter sheets alternate with permeate and retentate sheets, and as a liquid to be filtered flows across the filter sheets, impermeate (non-permeating) species, e.g., solids or high-molecular-weight species of diameter larger than the filter sheet's pore size(s), are retained and enter the retentate flow, and the liquid along with any permeate species diffuse through the filter sheet and enter the permeate flow. Each filter plate has on the inlet side, a transverse liquid feed trough and on the outlet side, a liquid collection trough. Between the liquid feed trough and the liquid collection trough is a plurality of parallel partitions that define subchannels and are of a lesser height than a wall that circumscribes the flow channel that is between the two troughs. In a preferred embodiment of the present invention, such cross-flow filtration module comprises a permeate collection and discharge arrangement, a feed inlet, a retentate outlet, and multiple fluid-flow sub-channels that may for example be equidistant to the inlet and the outlet.

The filter unit may be employed in stacked arrays to form a stacked cassette filter assembly in which the base sequence of retentate sheet (R), filter sheet (F), permeate sheet (P), filter sheet (F), and retentate sheet (R) may be repeated in the sequence of sheets in a filter assembly, the sheets shown in FIG. 13. Thus, the filter cassette of a desired total mass transfer area is readily formed from a stack of the repetitive sequences. In all repetitive sequences, except for a single unit sequence, the following relationship is observed: where X is the number of filter sheets, 0.5X−1 is the number of interior retentate sheets, and 0.5X is the number of permeate sheets, with two outer retentate sheets being provided at the outer extremities of the stacked sheet array.

The filter sheets, and the retentate and permeate sheets employed therewith, may be formed of any suitable materials of construction, including, for example, polymers, such as polypropylene, polyethylene, polysulfone, polyethersulfone, polyetherimide, polyimide, polyvinylchloride, polyester, etc.; nylon, silicone, urethane, regenerated cellulose, polycarbonate, cellulose acetate, cellulose triacetate, cellulose nitrate, mixed esters of cellulose, etc.; ceramics, e.g., oxides of silicon, zirconium, and/or aluminum; metals such as stainless steel; polymeric fluorocarbons such as polytetrafluoroethylene; and compatible alloys, mixtures and composites of such materials.

Preferably, the filter sheets and the retentate and permeate sheets are made of materials which are adapted to accommodate high temperatures and chemical sterilants, so that the interior surfaces of the filter may be steam sterilized and/or chemically sanitized for regeneration and reuse, as “steam-in-place” and/or “sterilizable in situ” structures, respectively. Steam sterilization typically may be carried out at temperatures on the order of from about 121° C. to about 130° C., at steam pressures of 15-30 psi, and at a sterilization exposure time typically on the order of from about 15 minutes to about 2 hours, or even longer. Alternatively, the entire cassette structure may be formed of materials which render the cassette article disposable in character.

Thus, the filtration unit comprises a multilaminate array of sheet members of generally rectangular and generally planar shape with main top and bottom surfaces, wherein the sheet members include in sequence in the array a first retentate sheet, a first filter sheet, a permeate sheet, and second filter sheet, and a second retentate sheet, wherein each of the sheet members in the array has at least one inlet basin opening at one end thereof, and at least one outlet basin opening at an opposite end thereof, with at least one permeate passage opening at longitudinal side margin portions of the sheet members; each of the first and second retentate sheets having at least one channel opening therein, wherein each channel opening extends longitudinally between the inlet and outlet basin openings of the sheets in the array and is open through the entire thickness of the retentate sheet, and with each of the first and second retentate sheets being bonded to an adjacent filter sheet about peripheral end and side portions thereof, with their basin openings and permeate passage openings in register with one another, and arranged to permit flow of filtrate through the channel openings of the retentate sheet between the inlet and outlet basin openings to permit permeate flow through the filter sheet to the permeate sheet to the permeate passage openings.

According to one embodiment of the present invention, a cross-flow filtration module with uniform geometry is utilized for conducting the membrane separation. The phrase “uniform geometry” is defined herein as the geometric structure of a cross-flow filtration module, characterized by at least one permeate flow passage, at least one inlet, at least one outlet, and multiple fluid-flow sub-channels that are of substantially equal length between the inlet and the outlet.

Notably, the cross-flow filtration modules with sub-channels that are equidistant to the inlet and outlet of said modules are employed for membrane separation. Moreover, such cross-flow filtration modules are characterized by optimal channel height, optimal transmembrane pressure, optimal membrane pore size and pore structure, optimal membrane chemistry, etc., which characteristics are selected in order to achieve the best combination of product quality and production yield.

In operation of the stacked filter plate assembly, liquid is introduced via the liquid inlet port. The liquid enters the liquid feed trough and is laterally distributed from the medial portion of the feed trough into the subchannels and toward its outer extremities in a highly uniform flow over the full areal extent of the sheet filter elements. This structure results in an increased solids filtration capacity and extended operation time and thus a higher microbial or virus yield may be obtained before the filter must be regenerated or changed.

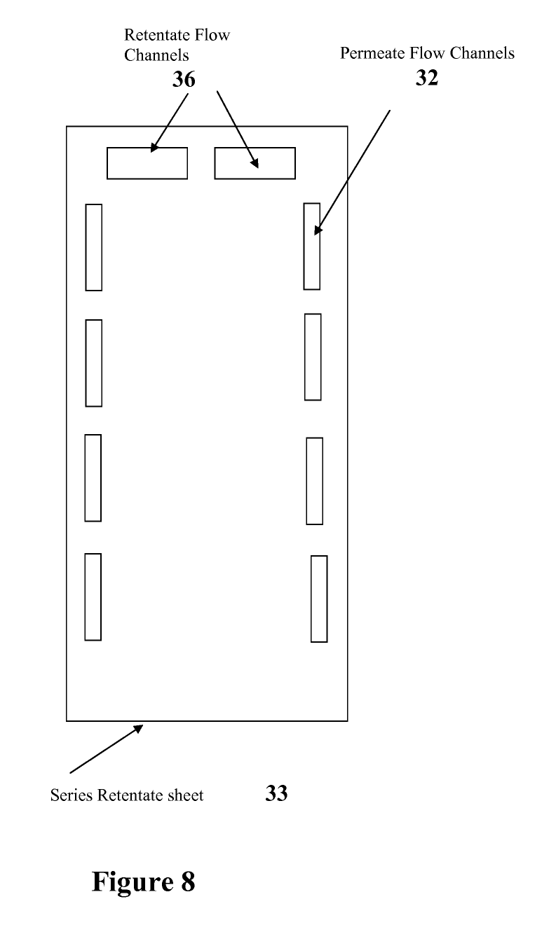

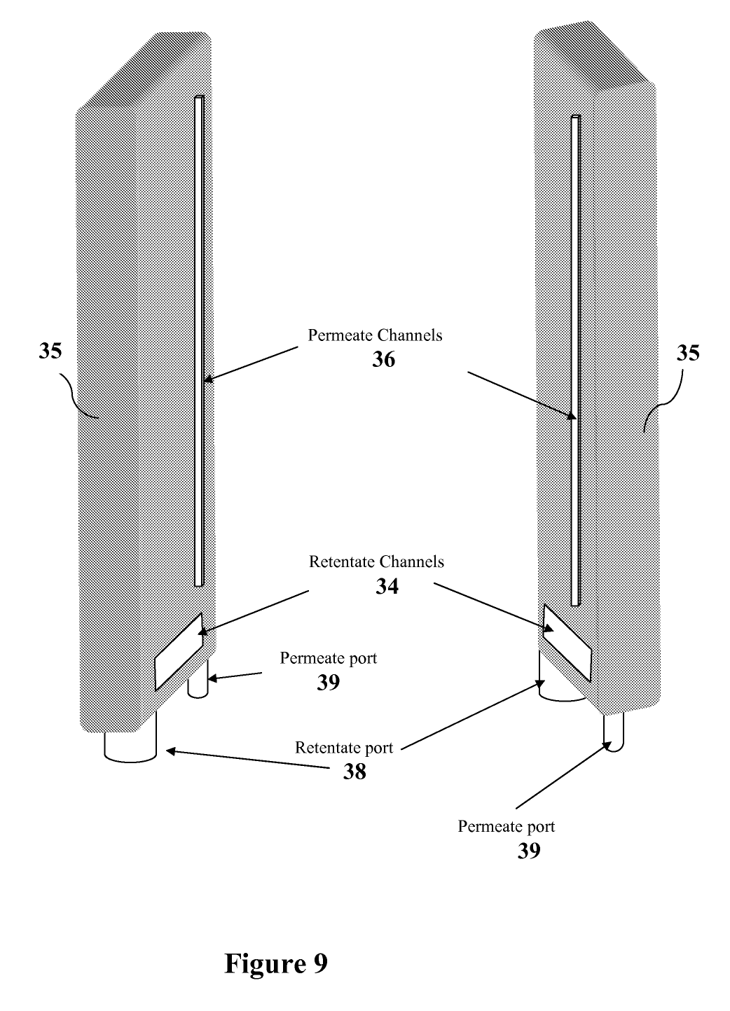

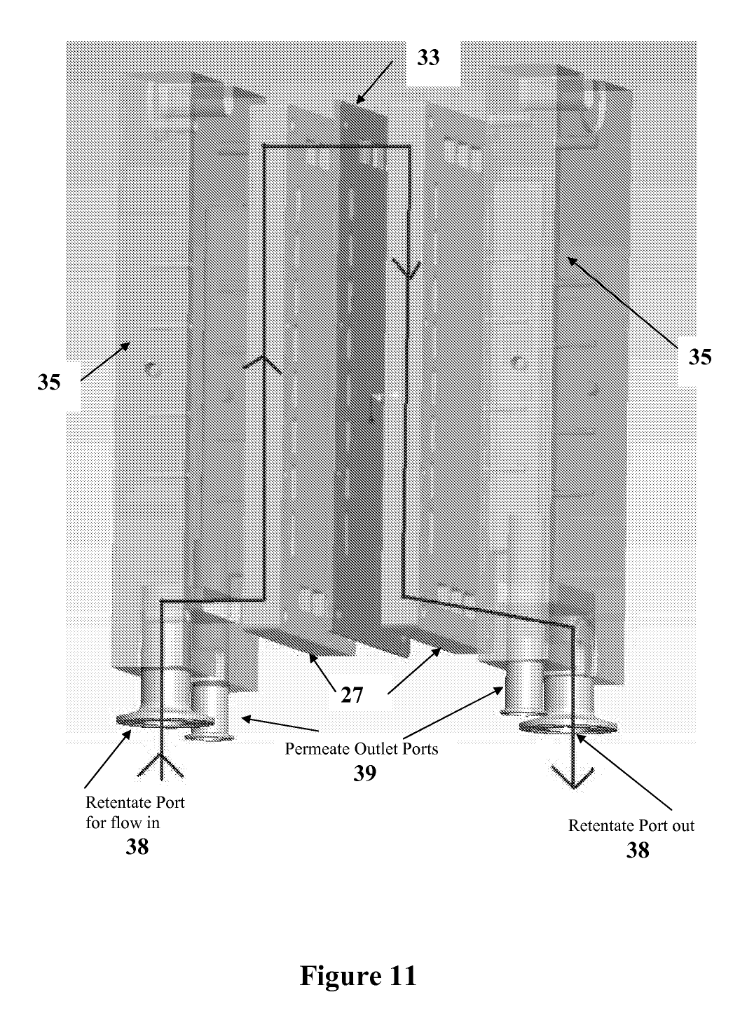

FIGS. 9 and 11 illustrate an alternative setup showing a first cross-flow filtration stack, as shown in FIG. 13, used in a series-flow configuration wherein the filter end plates of FIG. 9 secure the sheets of FIGS. 8 and 13 therebetween and the source liquid is diverted and moved along the longitudinal length of the separator and center sheet 33 of FIG. 8 until it passes through the retentate channels 36 and then into a second cross-flow filtration stack for movement therethrough and existing therefrom as shown in FIG. 11. Notably the separator and center sheet 33 includes only permeate flow channels along the longitudinal axis of the sheet and only one set of retentate flow channels on only one end and positioned normal to the permeate flow channels. As fluid enters the first filtration array 27 the fluid is directed in one direction and when it reaches the one set of retentate flow channels, the fluid as the ability to move into a second filtration array as shown in FIG. 11.

The filter end plates 35 as shown in FIG. 9 comprise a pair of rectangular or square base members wherein each base plate member comprises a first face side and a second face side, a first end and second end side positioned along the longitudinal axis of the base member, perpendicular to the first and second face sides; and a third end and fourth end side positioned normal to the first end and second end side, wherein the first face side comprises a permeate channel 36 and a retentate channel 34, wherein the permeate channel is positioned within and along the longitudinal axis of the first face side of the base member positioned near the first or second end side and the retentate channel is positioned within the first face side of the base member and normal to the permeate channel, wherein the third end side comprises a permeate port 39 in fluid communication with the permeate channel and the third or fourth end side comprises a retentate port 38 in fluid communication with the retentate channel.

The filter end plates and may be formed of any suitable materials of construction, including plastics such as polypropylene, polyethylene, polysulfone, polyimides, etc.; ceramics; metals such as stainless steels; and polymeric fluorocarbons such as polytetrafluoroethylene. Preferably the materials used are capable of withstanding sterilization for regeneration and reuse such as by high-temperatures, steam sterilization and/or chemical sanitization. Thus, the foraminous support may comprise a sintered ceramic material, e.g., of alumina, zirconia, etc., having an internal network of interconnected voids with an average void passage diameter on the order of about 1 micron.

Direct flow/dead end filtration may also be used as the filtration device and may included any filters that provide for moving the feed stream perpendicularly to the membrane and purifying the source liquid as it passes through the membrane (filtrate). Particulates and aggregates remain behind as filter cake. Commercially available units are available from GE Healthcare, including the ULTA™ family of normal flow filtration products.

All of the steps for culturing cells or microorganisms, between the initial inoculation of the bioreactor and the removal of the target substance from the separation device are preferably completed under conditions where all control and monitoring is completed by a single monitoring system and accessed through the computer port. Importantly, if the system is being used to generate viruses, all viable viruses being completely contained within the system. All components of the bioreactor vessel, separation device and process lines, meters, sensors can be disposable, and thus, minimizing exposure to lab staff or requirements for sterilization. All sampling, monitoring, and medium adjustments may be performed automatically and aseptically. When the appropriate amount of time has elapsed to yield the desired target concentration, the system may be automatically changed from the cell growth to the product harvest phase by appropriate valve means adjustments, activated and implemented by the software within the single source computer.

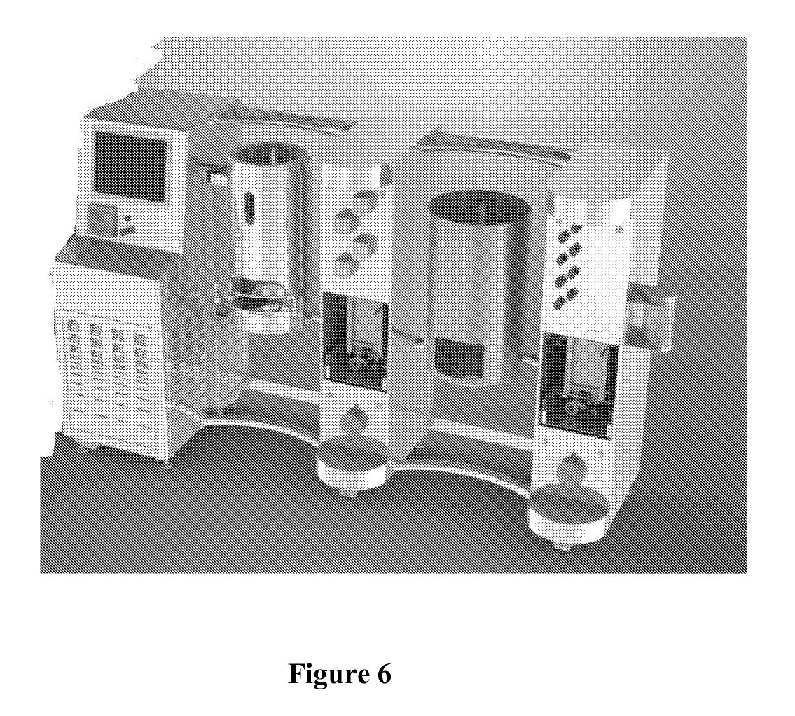

Notably, the above described system can easily adapt additional filtration or chromatography units by using the “plug and play” methodology because the operation and control of the different units occurs within a single system which is different from current systems that require multiple individual units that cannot cross communication to perform the culturing and separating processes of the present invention. FIG. 6 illustrates a system wherein the additional components are added when need with immediate communication to the central operating system.

That which is claimed is:

1. A culturing and separating system comprising:
a disposable container for housing biomaterials for processing, wherein the disposable container is held in support structure;
a separating unit in fluid communication with the disposable container, wherein the separating unit comprises at least one disposable filtration unit; and
a monitoring means for operating and controlling conditions in the bioreactor and separation unit.

2. The system according to claim 1, wherein the disposable contain further comprises at least one input port, at least one exhaust port, at least one harvest port, and one or more sensors for sensing one or more parameters of the biomaterials in the disposable container.

3. The system according to claim 2, wherein the parameters are temperature, dissolved oxygen, pH, tank level, agitation speed, levels of nutrients, flow rate of gas or fluids, inlet and outlet pressure, conductivity, turbidity, or UV radiation.

4. The system according to claim 1, wherein the disposable container hold from about 5 to 5000 liters of medium.

5. The system according to claim 1, wherein the disposable container is a liner within the support structure or bag.

6. The system according to claim 5, wherein the liner or bag is fabricated from a polymeric or cellulose containing material.

7. The system according to claim 1, wherein the disposable filtration unit comprises at least one stacked cassette filter assembly comprising a sequence of retentate sheet, filter sheet, permeate sheet, filter sheet, and retentate sheet.

8. The system according to claim 7, wherein the stacked cassette filter assembly is positioned between two filter end plates, wherein each filter endplate comprises a retentate port and a permeate port.

9. The system according to claim 8, wherein the filter end plates comprises a rectangular or square member comprising a permeate channel and a retentate channel, wherein the permeate channel is positioned within and along the longitudinal axis of the end plate and the retentate channel is positioned normal to the permeate channel, wherein the permeate channel is in fluid communication with the permeate port and the retentate channel is in fluid communication with the retentate port.

10. The system according to claim 7, wherein the system comprises two stacked cassette filter assembly and a separator sheet positioned therebetween.

11. A method of producing and separating a target substance comprising the steps of:
(a) introducing into a bioreactor a cell or micro-organism culture and culture medium fluid;
(b) maintaining the bioreactor and cells or microorganism under conditions to assure the expression of the target substance;
(c) moving a portion of the fluid, including cells or microorganisms and the target substance, through a separation device positioned in fluid communication with the bioreactor and separating therein at least some of the target substance from the fluid while allowing the remaining moving fluid and cells to pass through the separation device for return to the bioreactor; wherein the target substance separated from the fluid is removed to a collection vessel; and
(d) operating and controlling the system and processes therein with a single computer having the ability to control and adjust parameters within all the components of the system.

12. The method according to claim 11, wherein the bioreactor comprises a structural frame for holding a disposable container and the separation device is a disposable cross-flow filtration filter.

13. The method according to claim 12, further comprising additional disposable units selected from the group consisting of pump head, flow meter, pressure transducer, process lines and connectors between the bioreactor and separation unit.

14. The method according to claim 11, wherein the bioreactor comprises a disposable container, wherein the disposable container is held in support structure.

15. The method according to claim 11, wherein the disposable contain further comprises at least one input port, at least one exhaust port, at least one harvest port, and one or more sensors for sensing one or more parameters of the biomaterials in the disposable container.

16. The method according to claim 15, wherein the parameters are temperature, dissolved oxygen, pH, tank level, agitation speed, levels of nutrients, flow rate of gas or fluids, inlet and outlet pressure, conductivity, turbidity, or UV radiation.

17. The method according to claim 11, wherein the disposable container is a liner within the support structure or bag and wherein the liner or bag is fabricated from a polymeric or cellulose containing material.

18. The method according to claim 11, wherein the a separation device is a disposable filtration unit comprising at least one stacked cassette filter assembly comprising a sequence of retentate sheet, filter sheet, permeate sheet, filter sheet, and retentate sheet.

19. The method according to claim 18, wherein the stacked cassette filter assembly is positioned between two filter end plates, wherein each filter endplate comprises a retentate port and a permeate port.

20. The method according to claim 19, wherein the filter end plates comprises a rectangular or square member comprising a permeate channel and a retentate channel, wherein the permeate channel is positioned within and along the longitudinal axis of the end plate and the retentate channel is positioned normal to the permeate channel, wherein the permeate channel is in fluid communication with the permeate port and the retentate channel is in fluid communication with the retentate port.

21. The method according to claim 20, wherein the system comprises two stacked cassette filter assembly and a separator sheet positioned therebetween.

22. An integrated system comprising a bioreactor vessel in fluid communication with at least one separation unit and under the control of a Human-Machine Interphase (HMI) for controlling processes during the culturing of cells or microorganisms in a culture medium and separating any target substance therefrom.

23. The integrated system according to claim 22, wherein the separation unit is a filtration unit, a chromatography unit or a combination thereof.

24. The integrated system according to claim 22, wherein the HMI comprises integrating methods for operating and controlling the bioreactor and filtration and/or chromatography units.

25. The integrated system according to claim 22, wherein the bioreactor comprises a frame structure for holding a disposable container for culturing microorganisms or cells therein.
